(19) United States
(12) Patent Application Publication
(10) Pub. No.: US 2021/0244778 A1
(43) Pub. Date: Aug. 12, 2021

(54) BIFIDOBACTERIA FOR REDUCING FOOD, ENERGY AND/OR FAT INTAKE

(71) Applicant: DUPONT NUTRITION BIOSCIENCES APS, Copenhagen (DK)

(72) Inventors: Lotta Stenman, Kantvik (FI); Sampo Lahtinen, Lohja (FI)

(21) Appl. No.: 17/077,770

(22) Filed: Oct. 22, 2020

Related U.S. Application Data
(63) Continuation of application No. 16/090,975, filed on Oct. 3, 2018, now abandoned.

(30) Foreign Application Priority Data
Apr. 14, 2016 (EP) 16165379.5

(51) Int. Cl. A61K 35/745; A23L 33/00; C12N 1/20; A61P 3/00; A23L 33/26; A23L 33/135; A61K 47/36; C12R 1/01

(52) U.S. Cl. CPC A61K 35/745; A23L 33/30; C12N 1/20; C12N 1/205; A61P 3/00; A23L 33/26; A23V 2002/00; A61K 47/36; C12R 2001/01; A23V 2200/3204; A23Y 2300/49; A23Y 2300/21; A23V 2200/332; A23L 33/135

(57) ABSTRACT

This invention relates to the use of a bacterium of the genus Bifidobacterium, particularly, but not exclusively, a bacterium of the Bifidobacterium animalis ssp. lactis strain 420 (B420) for use in reducing food, energy and/or fat intake.

### FIELD OF THE INVENTION

This invention relates to new uses of a bacterium of the genus Bifidobacterium, particularly, but not exclusively, a bacterium of the Bifidobacterium animalis subspecies lactis strain 420 (B420). This invention also relates to food products, dietary supplements and pharmaceutically acceptable formulations containing said bacterium.

### BACKGROUND OF THE INVENTION

Regulation of energy balance is critical for the survival of an organism. When nutrients are freely available they are stored to account for low energy intake during times of scarcity. In a normal state the brain, together with energy storage tissues, regulates energy balance by reducing energy intake when energy stores are congested. If the energy storage machinery is disturbed, the brain is no longer able to maintain energy balance. This could lead to inability to maintain adequate energy intake, often manifested as wilting in elderly populations, or to excess storage of energy in adipose tissue and even obesity.

Body mass index (BMI) is a simple index of weight-for-height that is commonly used to classify body weight status in adults. It is defined as a person's weight in kilograms divided by the square of his height in meters (kg/m2).

The World Health Organisation (WHO) definition is: a BMI below 18.5 is underweight; a BMI between 18.5 and 24.99 is normal weight; a BMI greater than or equal to 25 is overweight; a BMI greater than or equal to 30 is obesity.

Overweight and obesity are defined as abnormal or excessive fat accumulation. Underweight is defined as a body weight that is too low to maintain normal bodily functions. Both underweight and overweight may lead to disturbances in metabolic functions, such as hormonal signalling.

Once considered a high-income country problem, overweight and obesity are now on the rise in low- and middle-income countries, particularly in urban settings. In developing countries with emerging economies (classified by the World Bank as lower- and middle-income countries) the rate of increase of childhood overweight and obesity has been more than 30% higher than that of developed countries.

Overweight and obesity are linked to more deaths worldwide than underweight. Most of the world's population live in countries where overweight and obesity kill more people than underweight (this includes all high-income and most middle-income countries). The fundamental cause of obesity and overweight is an energy imbalance between calories consumed and calories expended.

The most common consequences of overweight and obesity are diseases such as: cardiovascular diseases (mainly heart disease and stroke), which were the leading cause of death in 2012; diabetes; musculoskeletal disorders (especially osteoarthritis—a highly disabling degenerative disease of the joints); some cancers (endometrial, breast, and colon).

The risk for these diseases increases with an increase in BMI.

Childhood obesity is associated with a higher chance of obesity, premature death and disability in adulthood. But in addition to increased future risks, obese children experience breathing difficulties, increased risk of fractures, hypertension, early markers of cardiovascular disease, insulin resistance and psychological effects.

Overweight and obesity, as well as their related diseases, are largely preventable, and the food industry can play a significant role in the fight against obesity.

It is well known that dysfunctional energy regulation may lead to a variety of metabolic disorders, including obesity. The connections between gut microbiota, energy homeostasis, and the pathogenesis of metabolic disorders are now well-established (Daisuke et al., Front Endocrinol (Lausanne). 2014; 5: 81).

The recent publication “Neuronal regulation of Energy Homeostasis: Beyond the Hypothalamus and feeding”, Michael J Waterson et al., Cell Metabolism 22, Dec. 1, 2015, stresses the importance of the brain in maintaining energy homeostasis and its relation with obesity and other metabolic diseases.

### SUMMARY OF THE INVENTION

In one aspect, the invention concerns a bacterium of the genus bifidobacterium or a mixture thereof for use in reducing food, energy and/or fat intake in a mammal.

In particular, the invention concerns a bacterium of the genus Bifidobacterium or a mixture thereof as a probiotic for use in reducing food, energy and/or fat intake in a mammal.

In particular, the invention concerns a bacterium of the Bifidobacterium Animalis ssp. lactis strain 420 (B420).

In another aspect, the invention concerns a bacterium of the genus Bifidobacterium or a mixture thereof for use in therapy to reduce food, energy and/or fat intake in a mammal.

In particular, the invention concerns a bacterium of the genus Bifidobacterium or a mixture thereof for use in therapy to reduce food, energy and/or fat intake in a mammal, wherein the bacterium of the genus Bifidobacterium or a mixture thereof is a probiotic.

In particular, the invention concerns a bacterium of the genus Bifidobacterium or a mixture thereof for use in therapy to reduce food, energy and/or fat intake in a mammal, wherein the bacterium of the genus Bifidobacterium is the Bifidobacterium of the Bifidobacterium animalis ssp. lactis strain 420 (B420).

In a further aspect, the invention provides use of a bacterium of the genus Bifidobacterium or a mixture thereof for reducing food, energy and/or fat intake in a mammal.

In particular, the invention provides use of a bacterium of the genus Bifidobacterium or a mixture thereof as a probiotic for reducing food, energy and/or fat intake in a mammal.

In particular, the invention provides use of a bacterium of the Bifidobacterium animalis ssp. lactis strain 420 (B420).

In a further aspect, the present invention provides a non-therapeutic use of a bacterium of the genus Bifidobacterium or a mixture thereof for reducing food, energy and/or fat intake in a mammal.

In particular, the present invention provides a non-therapeutic use of a bacterium of the genus Bifidobacterium or a mixture thereof as probiotics for reducing food, energy and/or fat intake in a mammal.

In particular, the non-therapeutic use of the bacterium of the genus Bifidobacterium concerns the Bifidobacterium of the Bifidobacterium animalis ssp. lactis strain 420 (B420).

In a yet further aspect, the invention comprises a method for reducing food, energy and/or fat intake comprising administering to a mammal a bacterium of the genus Bifidobacterium or a mixture thereof, wherein the administration of the bacterium of the genus Bifidobacterium reduces the food, energy and/or fat intake in the mammal.

In particular, the invention comprises a method for reducing food, energy and/or fat intake comprising administering to a mammal a bacterium of the genus Bifidobacterium or a mixture thereof as a probiotic, wherein the administration of the probiotic bacterium reduces the food, energy and/or fat intake in the mammal.

In particular, the invention comprises a method for reducing food, energy and/or fat intake comprising administering to a mammal a Bifidobacterium of the subspecies Bifidobacterium animalis ssp. lactis strain 420 (B420), wherein the administration of the Bifidobacterium of the Bifidobacterium animalis ssp. lactis strain 420 (B420) reduces the food, energy and/or fat intake in the mammal.

In a yet further aspect, the invention comprises a non-therapeutic method for reducing food, energy and/or fat intake comprising administering to a mammal a bacterium of the genus Bifidobacterium or a mixture thereof, wherein the administration of the bacterium of the genus Bifidobacterium reduces the food, energy and/or fat intake in the mammal.

In particular, the invention comprises a non-therapeutic method for reducing food, energy and/or fat intake comprising administering to a mammal a bacterium of the genus Bifidobacterium or a mixture thereof as a probiotic, wherein the administration of the probiotic bacterium reduces the food, energy and/or fat intake in the mammal.

In particular, the invention comprises a non-therapeutic method for reducing food, energy and/or fat intake comprising administering to a mammal a Bifidobacterium of the subspecies Bifidobacterium animalis ssp. lactis strain 420 (B420), wherein the administration of the Bifidobacterium of the Bifidobacterium animalis ssp. lactis strain 420 (B420) reduces the food, energy and/or fat intake in the mammal.

In a still further aspect, the invention comprises use of a bacterium of the genus Bifidobacterium or a mixture thereof for the manufacture of a food product, a dietary supplement or a pharmaceutically acceptable formulation for reducing food, energy and/or fat intake in a mammal.

In a particular aspect, the invention comprises the use of a bacterium of the genus Bifidobacterium or a mixture thereof as a probiotic for the manufacture of a food product, a dietary supplement or a pharmaceutically acceptable formulation for reducing food, energy and/or fat intake in a mammal.

In a particular aspect, the invention comprises the use of a Bifidobacterium of the Bifidobacterium animalis ssp. lactis strain 420 (B420) or a mixture thereof for the manufacture of a food product, a dietary supplement or a pharmaceutically acceptable formulation for reducing food, energy and/or fat intake in a mammal.

Advantages

It has surprisingly been found by the present inventors that treatment with a bacterium of the genus Bifidobacterium or a mixture thereof, especially the Bifidobacterium of the Bifidobacterium animalis ssp. lactis strain 420 (B420), shows a reduction in food, energy and/or fat intake in a mammal. This confers the potential for bacterium of the genus Bifidobacterium or a mixture thereof, especially the Bifidobacterium of the Bifidobacterium animalis ssp. lactis strain 420 (B420) to be useful in treating and/or preventing a number of obesity-related diseases, such as high blood pressure, diabetes, heart diseases, high cholesterol levels, cancer, infertility, among others, through a physiological mechanism that reduces the energy intake in a mammal.

Without wishing to be bound by theory, it is believed that the Bifidobacterium of the present invention, when used alone or in combination with one with one or more fibres and/or prebiotics, has an effect on the gut microbiota, on the brain and on the energy homeostasis, helping control obesity and other metabolic diseases.

### DETAILED DISCLOSURE OF THE INVENTION

Bacteria

The bacterium used in the present invention is selected from a bacterium of the genus Bifidobacterium or a mixture thereof. Preferably the Bifidobacterium to be used in the present invention is a Bifidobacterium which is generally recognised as safe and, which is preferably GRAS approved. Generally recognized as safe (GRAS) is an American Food and Drug Administration (FDA) designation that a chemical or substance added to food is considered safe by experts, and so is exempted from the usual Federal Food, Drug, and Cosmetic Act (FFDCA) food additive tolerance requirements.

In one embodiment, the present invention relates to a bacterium of the genus Bifidobacterium or a mixture thereof for use in reducing food, energy and/or fat intake in a mammal.

In another embodiment, the present invention relates to use of a bacterium of the genus Bifidobacterium or a mixture thereof for reducing food, energy and/or fat intake in a mammal

In a further embodiment, the present invention relates to a method for reducing food, energy and/or fat intake comprising administering to a mammal a bacterium of the genus Bifidobacterium or a mixture thereof, wherein the administration of the bacterium of the genus Bifidobacterium reduces the food, energy and/or fat intake in the mammal.

In yet a further embodiment, the present invention relates to use of a bacterium of the genus Bifidobacterium or a mixture thereof for the manufacture of a food product, a dietary supplement or a pharmaceutically acceptable formulation for reducing food, energy and/or fat intake in a mammal.

The bacterium may be used in any form capable of exerting the effects described herein. For example, the bacteria may be viable, dormant, inactivated or dead bacteria. Preferably, the bacteria are viable bacteria.

The bacteria may comprise whole bacteria or may comprise bacterial components. Examples of such components include bacterial cell wall components such as peptidoglycan, bacterial nucleic acids such as DNA and RNA, bacterial membrane components, and bacterial structural components such as proteins, carbohydrates, lipids and combinations of these such as lipoproteins, glycolipids and glycoproteins.

The bacteria may also or alternatively comprise bacterial metabolites. In the present specification the term “bacterial metabolites” includes all molecules produced or modified by the (probiotic) bacteria as a result of bacterial metabolism during growth, survival, persistence, transit or existence of bacteria during the manufacture of the probiotic product and storage and during gastrointestinal transit in a mammal. Examples include all organic acids, inorganic acids, bases, proteins and peptides, enzymes and co-enzymes, amino acids and nucleic acids, carbohydrates, lipids, glycoproteins, lipoproteins, glycolipids, vitamins, all bioactive compounds, metabolites containing an inorganic component, and all small molecules, for example nitrous molecules or molecules containing a sulphurous acid.

Preferably the bacteria comprise whole bacteria, more preferably whole viable bacteria.

Preferably, the Bifidobacterium used in accordance with the present invention is one which is suitable for human and/or animal consumption. A skilled person will be readily aware of specific species and or strains of Bifidobacteria from within the genera described herein which are used in the food and/or agricultural industries and which are generally considered suitable for human and/or animal consumption.

In the present invention, the Bifidobacterium used may be of the same type (species and strain) or may comprise a mixture of species and/or strains.

Suitable bacteria are selected from the species Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

Preferably, the Bifidobacterium used in the present invention is of the species Bifidobacterium animalis. More preferably, the Bifidobacferium used in the present invention is of the Bifidobacterium animalis ssp. lactis.

In a particularly preferred embodiment, the bacteria used in the present invention are Bifidobacterium animalis ssp. lactis strain 420 (B420). This strain is commercially available from DuPont Nutrition Biosciences ApS.

This strain of Bifidobacterium animalis ssp. lactis has also been deposited under the reference DGCC420 by DuPont Nutrition Biosciences ApS, of Langebrogade 1, DK-1411 Copenhagen K, Denmark, in accordance with the Budapest Treaty on 30 Jun. 2015 at the Leibniz-Institut Deutsche Sammlung von Mikroorganismen and Zellkulturen GmbH (DSMZ), Inhoffenstrasse 7B, 38124 Braunschweig, Germany, where it is recorded under registration number DSM 32073. It is requested that the biological material shall be made available only by the issue of a sample to an expert nominated by the requester.

In one embodiment, the bacterium used in the present invention is a probiotic bacterium. In this specification the term ‘probiotic bacterium’ is defined as covering any non-pathogenic bacterium which, when administered live in adequate amounts, confer a health benefit on the host. These probiotic strains generally have the ability to survive the passage through the upper part of the digestive tract. They are non-pathogenic, non-toxic and exercise their beneficial effect on health on the one hand via ecological interactions with the resident flora in the digestive tract, and on the other hand via their ability to influence the immune system in a positive manner via the “GALT” (gut-associated lymphoid tissue). Depending on the definition of probiotics, these bacteria, when given in a sufficient number, have the ability to progress live through the intestine, however they do not cross the intestinal barrier and their primary effects are therefore induced in the lumen and/or the wall of the gastrointestinal tract. They then form part of the resident flora during the administration period. This colonization (or transient colonization) allows the probiotic bacteria to exercise a beneficial effect, such as the repression of potentially pathogenic micro-organisms present in the flora and interactions with the immune system of the intestine.

In preferred embodiments, the bacterium used in the present invention is a probiotic Bifidobacterium.

Dosage

The Bifidobacterium, such as a strain of Bifidobacterium animalis ssp. lactis, for example Bifidobacterium animalis ssp. lactis strain 420 (B420), used in accordance with the present invention may comprise from 10⁶ to 10¹² CFU of bacteria/g of support, and more particularly from 10⁸ to 10¹² CFU of bacteria/g of support, preferably 10⁹ to 10¹² CFU/g for the lyophilized form.

Suitably, the Bifidobacterium, such as a strain of Bifidobacterium animalis ssp. lactis, for example Bifidobacterium animalis ssp. lactis strain 420 (B420), may be administered at a dosage of from about 10⁶ to about 10¹² CFU of microorganism/dose, preferably about 10⁸ to about 10¹² CFU of microorganism/dose. By the term “per dose” it is meant that this amount of microorganism is provided to a subject either per day or per intake, preferably per day. For example, if the microorganism is to be administered in a food product, for example in a yoghurt, then the yoghurt will preferably contain from about 10⁸ to 10¹² CFU of the microorganism. Alternatively, however, this amount of microorganism may be split into multiple administrations each consisting of a smaller amount of microbial loading—so long as the overall amount of microorganism received by the subject in any specific time, for instance each 24-hour period, is from about 10⁶ to about 10¹² CFU of microorganism, preferably 10⁸ to about 10¹² CFU of microorganism.

In accordance with the present invention an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose.

In one embodiment, preferably the Bifidobacterium, such as a strain of Bifidobacterium animalis ssp. lactis, for example Bifidobacterium animalis ssp. lactis strain 420 (B420), may be administered at a dosage of from about 10⁶ to about 10¹² CFU of microorganism/day, preferably about 10⁸ to about 10¹² CFU of microorganism/day. Hence, the effective amount in this embodiment may be from about 10⁶ to about 10¹² CFU of microorganism/day, preferably about 10⁸ to about 10¹² CFU of microorganism/day.

CFU stands for “colony-forming units”. By ‘support’ is meant the food product, dietary supplement or the pharmaceutically acceptable formulation.

In one embodiment, the present invention relates to a bacterium of the genus Bifidobacterium or a mixture thereof, such as a strain of Bifidobacterium animalis ssp. lactis, for example Bifidobacterium animalis ssp. lactis strain 420 (B420), in the form of a food product, a dietary supplement or a pharmaceutically acceptable formulation.

Effects/Subjects/Medical Indications

The Bifidobacteria to which the present invention relates are administered to a mammal, including for example livestock (including cattle, horses, pigs and sheep), and humans. In some aspects of the present invention the mammal is a companion animal (including pets), such as a dog or a cat for instance. In some aspects of the present invention, the subject may suitably be a human.

The Bifidobacteria to which the present invention relates may be suitable for reducing food, energy intake in mammals.

Although birds and poultry, including chickens, are technically not mammals, the present invention may also be suitable for birds and any type of poultry, such as chickens.

The Bifidobacteria to which the present invention relates is also suitable for reducing fat intake in mammals.

The Bifidobacteria to which the present invention relates may also be suitable for reducing simultaneously food, energy and fat intake in mammals.

In this specification the term “reducing food intake and/or fat intake” refers to any administration of the Bifidobacteria according to the present invention and includes reduction of the number of calories absorbed by the mammal, birds or poultry.

In particular, the Bifidobacteria according to the present invention is suitable for mammals, birds and poultry ingesting a high-fat diet. This aspect is discussed in more detail below.

Diet

As noted above, subject mammals, birds or poultry treated with bacteria according to the present invention may ingest a high-fat diet while mitigating the metabolic consequences of their condition(s). In this specification the term ‘high-fat diet’ means a diet generally containing at least 20%, preferably at least 25%, such as at least 30%, for example at least 35%, such as at least 40%, for example at least 45%, such as at least 50%, for example at least 55%, such as at least 60%, for example at least 65%, such as at least 70%, for example at least 75%, such as at least 80%, for example at least 85%, such as at least 90% of calories from fat.

In some embodiments, mammals, birds or poultry treated with bacteria according to the present invention may ingest a low-carbohydrate diet during the course of the treatment. In this specification the term ‘low-carbohydrate diet’ means a diet generally containing no greater than 50%, such as no greater than 45%, for example no greater than 40%, such as no greater than 35%, for example no greater than 30%, such as no greater than 25%, for example no greater than 20%, such as no greater than 15%, for example no greater than 10%, such as no greater than 5%, for example no greater than 2%, such as no greater than 1%, for example no greater than 0.5%, such as no greater than 0.2% of calories from carbohydrate.

Compositions

While is it possible to administer Bifidobacferia alone according to the present invention (i.e. without any support, diluent or excipient), the Bifidobacteria are typically and preferably administered on or in a support as part of a product, in particular as a component of a food product, a dietary supplement or a pharmaceutical formulation. These products typically contain additional components well known to those skilled in the art.

Any product which can benefit from the composition may be used in the present invention. These include but are not limited to foods, particularly fruit conserves and dairy foods and dairy food-derived products, and pharmaceutical products. The Bifidobacteria may be referred to herein as “the composition of the present invention” or “the composition”.

Food

In one embodiment, the Bifidobacteria are employed according to the invention in a food product, such as a food supplement, a drink or a powder based on milk. Here, the term “food” is used in a broad sense and covers food for humans as well as food for animals (i.e. a feed). In a preferred aspect, the food is for human consumption.

The food may be in the form of a solution or as a solid, depending on the use and/or the mode of application and/or the mode of administration.

When used as, or in the preparation of, a food, such as functional food, the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

The composition can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped topping, low fat and light natural cheddar cheese, low fat Swiss style yoghurt, aerated frozen desserts, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

The term “dairy product” as used herein is meant to include a medium comprising milk of animal and/or vegetable origin. As milk of animal origin there can be mentioned cow's, sheep's, goat's or buffalo's milk. As milk of vegetable origin there can be mentioned any fermentable substance of vegetable origin which can be used according to the invention, in particular originating from soybeans, rice or cereals.

Still more preferably the food product employed according to the invention is a fermented milk or humanized milk.

For certain aspects, preferably the present invention may be used in connection with yoghurt production, such as fermented yoghurt drink, yoghurt, drinking yoghurt, cheese, fermented cream, milk based desserts and others.

Suitably, the composition can be further used as an ingredient in one or more of cheese applications, meat applications, or applications comprising protective cultures.

The present invention also provides a method of preparing a food or a food ingredient, the method comprising admixing the composition according to the present invention with another food ingredient.

Advantageously, the present invention relates to products that have been contacted with the composition of the present invention, and optionally with other components/ingredients, wherein the composition is used in an amount to be capable of improving the nutrition and/or health benefits of the product.

As used herein the term “contacted” refers to the indirect or direct application of the composition of the present invention to the product. Examples of the application methods which may be used, include, but are not limited to, treating the product in a material comprising the composition, direct application by mixing the composition with the product, spraying the composition onto the product surface or dipping the product into a preparation of the composition.

Where the product of the invention is a foodstuff, the composition of the present invention is preferably admixed with the product. Alternatively, the composition may be included in the emulsion or raw ingredients of a foodstuff. In a further alternative, the composition may be applied as a seasoning, glaze, colorant mixture, and the like.

For some applications, it is important that the composition is made available on or to the surface of a product to be affected/treated. This allows the composition to impart one or more of the following favourable characteristics: nutrition and/or health benefits.

The compositions of the present invention may be applied to intersperse, coat and/or impregnate a product with a controlled amount of a microorganism.

Preferably, the composition is used to ferment milk or sucrose fortified milk or lactic media with sucrose and/or maltose where the resulting media containing all components of the composition—i.e. said microorganism according to the present invention—can be added as an ingredient to yoghurt milk in suitable concentrations—such as for example in concentrations in the final product which offer a daily dose of 10⁶-10¹⁰ CFU. The microorganism according to the present invention may be used before or after fermentation of the yoghurt.

For some aspects the microorganisms according to the present invention are used as, or in the preparation of, animal feeds, such as livestock feeds, in particular poultry (such as chicken) feed, or pet food.

Advantageously, where the product is a food product, the Bifidobacteria should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

Food Ingredient

The composition of the present invention may be used as a food ingredient and/or feed ingredient.

As used herein the term “food ingredient” or “feed ingredient” includes a formulation which is or can be added to functional foods or foodstuffs as a nutritional supplement.

The food ingredient may be in the form of a solution or as a solid, depending on the use and/or the mode of application and/or the mode of administration.

Food Supplements

The composition of the present invention may be—or may be added to—dietary supplements, also referred to herein as food supplements.

Here, the term “dietary supplement” is a product intended for ingestion that contains a “dietary ingredient” intended to add further nutritional value to (supplement) the diet. A “dietary ingredient” may be one, or any combination, of the following substances: a vitamin, a mineral, an herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure that you get an adequate dietary intake of essential nutrients; others may help you reduce your risk of disease.

Functional Foods

The composition of the present invention may be—or may be added to—functional foods.

As used herein, the term “functional food” means food which is capable of providing not only a nutritional effect, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

Medical Food

In one embodiment, the bacterium of the present invention is in the form of a medical food. Preferably, the Bifidobacterium of the present invention, such as a strain of Bifidobacterium animalis ssp. lactis, for example Bifidobacterium animalis ssp. lactis strain 420 (B420) is in the form of a medical food.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Pharmaceutical

The composition of the present invention may be used as—or in the preparation of—a pharmaceutical formulation. Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry.

The pharmaceutical can be for therapeutic purposes—which may be curative or palliative or preventative in nature. The pharmaceutical may even be for diagnostic purposes.

A pharmaceutically acceptable formulation or support may be for example a formulation or support in the form of compressed tablets, tablets, capsules, ointments, suppositories or drinkable solutions. Other suitable forms are provided below.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a solution or as a solid—depending on the use and/or the mode of application and/or the mode of administration.

The Bifidobacteria of the present invention may be used as pharmaceutical ingredients. Here, the composition may be the sole active component or it may be at least one of a number (i.e. 2 or more) of active components.

The pharmaceutical ingredient may be in the form of a solution or as a solid—depending on the use and/or the mode of application and/or the mode of administration.

The Bifidobacteria may be used according to the present invention in any suitable form—whether when alone or when present in a combination with other components or ingredients. The Bifidobacteria used in the present invention may be referred to herein as “the composition”. Likewise, combinations comprising the composition of the present invention and other components and/or ingredients (i.e. ingredients—such as food ingredients, functional food ingredients or pharmaceutical ingredients) may be used in any suitable form.

The Bifidobacteria may be used according to the present invention in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the composition of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethrai fatty acid esters, hydroxymethylceilulose, polyvinylpyrrolidone, and the like.

Preferred excipients for the forms include lactose, starch, a cellulose, milk sugar or high molecular weight polyethylene glycols.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

The forms may also include gelatin capsules; fibre capsules, fibre tablets etc.; or even fibre beverages.

Further examples of form include creams. For some aspects the microorganism used in the present invention may be used in pharmaceutical and/or cosmetic creams such as sun creams and/or after-sun creams for example.

In one aspect, the composition according to the present invention may be administered in an aerosol, for example by way of a nasal spray, for instance for administration to the respiratory tract.

Medicament

In one embodiment, the bacterium of the present invention is in the form of a medicament.

The term “medicament” as used herein encompasses medicaments for both human and animal usage in human and veterinary medicine. In addition, the term “medicament” as used herein means any substance which provides a therapeutic and/or beneficial effect. The term “medicament” as used herein is not necessarily limited to substances which need Marketing Approval, but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

Prebiotics

In one embodiment, the bacterium of the present invention may contain one or more fibres and/or prebiotics.

Prebiotics are a category of functional food, defined as non-digestible food ingredients that beneficially affect the host by selectively stimulating the growth and/or activity of one or a limited number of bacteria (particularly, although not exclusively, probiotics, Bifidobacteria and/or lactic acid bacteria) in the colon, and thus improve host health. Typically, prebiotics are carbohydrates (such as oligosaccharides), but the definition does not preclude non-carbohydrates. The most prevalent forms of prebiotics are nutritionally classed as soluble fibres. To some extent, many forms of dietary fibres exhibit some level of prebiotic effect.

In one embodiment, a prebiotic is a selectively fermented ingredient that allows specific changes, both in the composition and/or activity in the gastrointestinal microflora that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 10 g/day, preferably 2 to 9 g/day, more preferably 3 to 8 g/day. In another embodiment, the prebiotic may be used according to the present invention in an amount of 5 to 50 g/day, preferably 10 to 25 g/day.

Examples of dietary sources of prebiotics include soybeans, inulin sources (such as Jerusalem artichoke, jicama, and chicory root), raw oats, unrefined wheat, unrefined barley and yacon.

Examples of suitable prebiotics include alginate, xanthan, pectin, locust bean gum (LBG), inulin, guar gum, galacto-oligosaccharide (GOS), fructo-oligosaccharide (FOS), polydextrose (i.e. Litesse®), lactitol, lactosucrose, soybean oligosaccharides, isomaltulose (Palatinose™), isomalto-oligosaccharides, gluco-oligosaccharides, xylooligosaccharides, manno-oligosaccharides, beta-glucans, cellobiose, raffinose, gentiobiose, melibiose, xylobiose, cyciodextrins, isomaltose, trehalose, stachyose, panose, pullulan, verbascose, galactomannans, and all forms of resistant starches.

A particularly preferred example of a prebiotic is polydextrose.

In some embodiments, a combination of Bifidobacterium and one or more fibres and/or prebiotics according to the present invention exhibits a synergistic effect in certain applications (i.e. an effect which is greater than the additive effect of the bacteria when used separately). Without wishing to be bound by theory, it is believed that such a combination is capable of selectively stimulating the growth and/or activity of the Bifidobacteria in the colon, and thus improving its effect and the host health.

In one embodiment, the Bifidobacterium or a mixture thereof used in the combination with one or more fibres and/or prebiotic is of the species Bifidobacterium animalis. More preferably, the Bifidobacterium or a mixture thereof used in the combination with one or more fibres and/or prebiotic is of the Bifidobacterium animalis ssp. lactis.

In a particularly preferred embodiment, the Bifidobacterium or a mixture thereof used in the combination with one or more fibres and/or prebiotic is of the Bifidobacterium animalis ssp. lactis strain 420 (B420).

Suitably, the fibre and/or prebiotic used in the combination is polydextrose.

In another embodiment, the fibre and/or prebiotic used in the combination is Litesse®.

In a further aspect, the invention comprises a food product comprising a Bifidobacterium or mixture thereof and one or more fibres and/or a prebiotic.

In a yet further aspect, the invention comprises a food product comprising the Bifidobacterium or mixture thereof of the species Bifidobacterium animalis and one or more fibres and/or a prebiotic.

In a yet further aspect, the invention comprises a food product comprising the Bifidobacterium or mixture thereof of the Bifidobacterium animalis ssp. lactis and one or more fibres and/or a prebiotic.

In a yet further aspect, the invention comprises a food product comprising the Bifidobacterium or mixture thereof of the Bifidobacterium animalis ssp. lactis strain 420 (B420) and one or more fibres and/or a prebiotic.

In a further aspect, the invention comprises a dietary supplement comprising a Bifidobacterium or mixture thereof and one or more fibres and/or a prebiotic.

In a yet further aspect, the invention comprises a dietary supplement comprising the Bifidobacterium or mixture thereof of the species Bifidobacterium animalis and one or more fibres and/or a prebiotic.

In a yet further aspect, the invention comprises a dietary supplement comprising the Bifidobacterium or mixture thereof of the Bifidobacterium animalis ssp. lactis and one or more fibres and/or a prebiotic.

In a yet further aspect, the invention comprises a dietary supplement comprising the Bifidobacterium or mixture thereof of the Bifidobacterium animalis ssp. lactis strain 420 (B420) and one or more fibres and/or a prebiotic.

In a further aspect, the invention comprises a pharmaceutically acceptable formulation comprising a Bifidobacterium or mixture thereof and one or more fibres and/or a prebiotic.

In a yet further aspect, the invention comprises a pharmaceutically acceptable formulation comprising the Bifidobacterium or mixture thereof of the species Bifidobacterium animalis and one or more fibres and/or a prebiotic.

In a yet further aspect, the invention comprises a pharmaceutically acceptable formulation comprising the Bifidobacterium or mixture thereof of the Bifidobacterium animalis ssp. lactis and one or more fibres and/or a prebiotic.

In a yet further aspect, the invention comprises a pharmaceutically acceptable formulation comprising the Bifidobacterium or mixture thereof of the Bifidobacterium animalis ssp. lactis strain 420 (B420) and one or more fibres and/or a prebiotic.

Specific numbered embodiments of the invention:

Embodiment 1. A bacterium of the genus Bifidobacterium or a mixture thereof for use in reducing food, energy and/or fat intake in a mammal.

Embodiment 2. The bacterium according to embodiment 1, wherein the bacterium of the genus Bifidobacterium or a mixture thereof is a probiotic.

Embodiment 3. The bacterium according to any one of embodiments 1 to 2, wherein the bacterium of the genus Bifidobacterium is a Bifidobacterium of the species Bifidobacterium animalis.

Embodiment 4. The bacterium according to any one of the embodiments 1 to 3, wherein the bacterium of the genus Bifidobacterium is a Bifidobacterium of the Bifidobacterium animalis ssp. lactis.

Embodiment 5. The bacterium according to any one of the embodiments 1 to 4, wherein the bacterium of the genus Bifidobacterium is the Bifidobacterium of the Bifidobacterium animalis ssp. lactis strain 420 (B420).

Embodiment 6. The bacterium according to any one embodiment 1 to 5, wherein the bacterium is used in combination with one or more fibres and/or prebiotics.

Embodiment 7. The bacterium according to embodiment 6, wherein the fibres and/or the prebiotic is polydextrose.

Embodiment 8. The bacterium according to any one of the preceding embodiments, wherein the bacterium is in the form of a food product, a dietary supplement or a pharmaceutically acceptable formulation.

Embodiment 9. The bacterium according to embodiment 8, wherein the pharmaceutically acceptable formulation is a medicament.

Embodiment 10. The bacterium according to embodiment 8, wherein the food product is a medical food product.

Embodiment 11. A bacterium of the genus Bifidobacterium or a mixture thereof for use in therapy to reduce food, energy and/or fat intake in a mammal.

Embodiment 12. The bacterium according to embodiment 11, wherein the bacterium of the genus Bifidobacterium or a mixture thereof is a probiotic.

Embodiment 13. The bacterium according to any one of embodiments 11 to 12, wherein the bacterium of the genus Bifidobacterium is a Bifidobacterium of the species Bifidobacterium animalis.

Embodiment 14. The bacterium according to any one of the embodiments 11 to 13, wherein the bacterium of the genus Bifidobacterium is a Bifidobacterium of the Bifidobacterium animalis ssp. lactis.

Embodiment 15. The bacterium according to any one of the embodiments 11 to 14, wherein the bacterium of the genus Bifidobacterium is the Bifidobacterium of the Bifidobacterium animalis ssp. lactis strain 420 (B420).

Embodiment 16. The bacterium according to any one embodiment 11 to 15, wherein the bacterium is used in combination with one or more fibres and/or prebiotics.

Embodiment 17. The bacterium according to embodiment 16, wherein the fibres and/or the prebiotic is polydextrose.

Embodiment 18. The bacterium according to any one of the preceding embodiments 11-17, wherein the bacterium is in the form of a food product, a dietary supplement or a pharmaceutically acceptable formulation.

Embodiment 19. The bacterium according to embodiment 18, wherein the pharmaceutically acceptable formulation is a medicament.

Embodiment 20. The bacterium according to embodiment 18, wherein the food product is a medical food product.

Embodiment 21. Use of a bacterium of the genus Bifidobacterium or a mixture thereof for reducing food, energy and/or fat intake in a mammal.

Embodiment 22. The use according to embodiment 21, wherein the bacterium of the genus Bifidobacterium or a mixture thereof is a probiotic.

Embodiment 23. The use according to any one of embodiments 21 to 22, wherein the bacterium of the genus Bifidobacterium is a Bifidobacterium of the species Bifidobacterium animalis.

Embodiment 24. The use according to any one of the embodiments 21 to 23, wherein the bacterium of the genus Bifidobacterium is a Bifidobacterium of the Bifidobacterium animalis ssp. lactis.

Embodiment 25. The use according to any one of the embodiments 21 to 24, wherein the bacterium of the genus Bifidobacterium is the Bifidobacterium of the Bifidobacterium animalis ssp. lactis strain 420 (B420).

Embodiment 26. The use according to any one embodiments 21 to 25, wherein the bacterium is used in combination with one or more fibres and/or prebiotics.

Embodiment 27. The use according to embodiment 26, wherein the fibres and/or prebiotic is polydextrose.

Embodiment 28. The use according to any one of the embodiments 21 to 27, wherein the bacterium is in the form of a food product, a dietary supplement or a pharmaceutically acceptable formulation.

Embodiment 29. The use according to embodiment 28, wherein the pharmaceutically acceptable formulation is a medicament.

Embodiment 30. The use according to embodiment 28, wherein the food product is a medical food product.

Embodiment 31. A non-therapeutic use of a bacterium of the genus Bifidobacterium or a mixture thereof for reducing food, energy and/or fat intake in a mammal.

Embodiment 32. The non-therapeutic use according to embodiment 31, wherein the bacterium of the genus Bifidobacterium or a mixture thereof is a probiotic.

Embodiment 33. The non-therapeutic use according to any one of embodiments 31 to 32, wherein the bacterium of the genus Bifidobacterium is a Bifidobacterium of the species Bifidobacterium animalis.

Embodiment 34. The non-therapeutic use according to any one of the embodiments 31 to 33, wherein the bacterium of the genus Bifidobacterium is a Bifidobacterium of the Bifidobacterium animalis ssp. lactis.

Embodiment 35. The non-therapeutic use according to any one of the embodiments 31 to 34, wherein the bacterium of the genus Bifidobacterium is the Bifidobacterium of the Bifidobacterium animalis ssp. lactis strain 420 (B420).

Embodiment 36. The non-therapeutic use according to any one embodiments 31 to 35, wherein the bacterium is used in combination with one or more fibres and/or prebiotics.

Embodiment 37. The non-therapeutic use according to embodiment 36, wherein the fibres and/or prebiotic is polydextrose.

Embodiment 38. The non-therapeutic use according to any one of the embodiments 31 to 37, wherein the bacterium is in the form of a food product, a dietary supplement or a pharmaceutically acceptable formulation.

Embodiment 39. The non-therapeutic use according to embodiment 38, wherein the pharmaceutically acceptable formulation is a medicament.

Embodiment 40. The non-therapeutic use according to embodiment 38, wherein the food product is a medical food product.

Embodiment 41. A method for reducing food, energy and/or fat intake comprising administering to a mammal a bacterium of the genus Bifidobacterium or a mixture thereof, wherein the administration of the bacterium of the genus Bifidobacterium reduces the food, energy and/or fat intake in the mammal.

Embodiment 42. The method according to embodiment 41, wherein the bacterium of the genus Bifidobacterium or a mixture thereof is a probiotic.

Embodiment 43. The method according to any one of embodiment 41 to 42, wherein the bacterium of the genus Bifidobacterium is a Bifidobacterium of the species Bifidobacterium animalis.

Embodiment 44. The method according to any one of the embodiment 41 to 43, wherein the bacterium of the genus Bifidobacterium is a Bifidobacterium of the Bifidobacterium animalis ssp. lactis.

Embodiment 45. The method according to any one of the embodiment 41 to 44, wherein the bacterium of the genus Bifidobacterium is a Bifidobacterium of the Bifidobacterium animalis ssp. lactis strain 420 (B420).

Embodiment 46. The method according to any one embodiment 41 to 45, wherein the bacterium is used in combination with one or more fibres and/or prebiotics.

Embodiment 47. The method according to embodiment 46, wherein the fibres and/or prebiotic is polydextrose.

Embodiment 48. The method according to any one of the embodiment 41 to 47, wherein the bacterium is in the form of a food product, a dietary supplement or a pharmaceutically acceptable formulation.

Embodiment 49. The method according to embodiment 48, wherein the pharmaceutically acceptable formulation is a medicament.

Embodiment 50. The method according to embodiment 48, wherein the food product is a medical food product.

Embodiment 51. A non-therapeutic method for reducing food, energy and/or fat intake comprising administering to a mammal a bacterium of the genus Bifidobacterium or a mixture thereof, wherein the administration of the bacterium of the genus Bifidobacterium reduces the food, energy and/or fat intake in the mammal.

Embodiment 52. The non-therapeutic method according to embodiment 51, wherein the bacterium of the genus Bifidobacterium or a mixture thereof is a probiotic.

Embodiment 53. The non-therapeutic method according to any one of embodiment 51 to 52, wherein the bacterium of the genus Bifidobacterium is a Bifidobacterium of the species Bifidobacterium animalis.

Embodiment 54. The non-therapeutic method according to any one of the embodiment 51 to 53, wherein the bacterium of the genus Bifidobacterium is a Bifidobacterium of the Bifidobacterium animalis ssp. lactis.

Embodiment 55. The non-therapeutic method according to any one of the embodiment 51 to 54, wherein the bacterium of the genus Bifidobacterium is a Bifidobacterium of the Bifidobacterium animalis ssp. lactis strain 420 (B420).

Embodiment 56. The non-therapeutic method according to any one embodiment 51 to 55, wherein the bacterium is used in combination with one or more fibres and/or prebiotics.

Embodiment 57. The non-therapeutic method according to embodiment 56, wherein the fibres and/or prebiotic is polydextrose.

Embodiment 58. The non-therapeutic method according to any one of the embodiment 51 to 57, wherein the bacterium is in the form of a food product, a dietary supplement or a pharmaceutically acceptable formulation.

Embodiment 59. The non-therapeutic method according to embodiment 58, wherein the pharmaceutically acceptable formulation is a medicament.

Embodiment 60. The non-therapeutic method according to embodiment 58, wherein the food product is a medical food product.

Embodiment 61. Use of a bacterium of the genus Bifidobacterium or a mixture thereof for the manufacture of a food product, a dietary supplement or a pharmaceutically acceptable formulation for reducing food, energy and/or fat intake in a mammal.

Embodiment 62. The use according to embodiment 61, wherein the bacterium of the genus Bifidobacterium or a mixture thereof is a probiotic.

Embodiment 63. The use according to any one of embodiment 61 to 62, wherein the bacterium of the genus Bifidobacterium is a Bifidobacterium of the species Bifidobacterium animalis.

Embodiment 64. The use according to any one of the embodiment 61 to 63, wherein the bacterium of the genus Bifidobacterium is a Bifidobacterium of the Bifidobacterium animalis ssp. lactis.

Embodiment 65. The use according to any one of the embodiment 61 to 64, wherein the bacterium of the genus Bifidobacterium is the Bifidobacterium of the Bifidobacterium animalis ssp. lactis strain 420 (B420).

Embodiment 66. The use according to any one embodiment 61 to 65, wherein the bacterium is used in combination with one or more fibres and/or prebiotics.

Embodiment 67. The use according to embodiment 66, wherein the fibres and/or prebiotic is polydextrose.

Embodiment 68. The use according to embodiment 61, wherein the pharmaceutically acceptable formulation is a medicament.

Embodiment 69. The use according to embodiment 61, wherein the food product is a medical food product.

### EXAMPLES

Materials and Methods

Clinical Study Design and Screening Criteria

The intervention was a double-blind, randomized, placebo-controlled, multi-centre parallel study, conducted according to Good Clinical Practice and the Declaration of Helsinki.

A cohort of 225 adults were selected from 263 overweight and obese adults at four research centres in southern Finland and randomized according to a 1:1:1:1 allocation to one of four groups:

1) Placebo (microcrystalline cellulose), 12 g/day;

2) Polydextrose, 12 g/day;

3) Probiotic B420 (Bifidobacterium animalis ssp. lactis 420), 10¹⁰ CFU/day; or

4) B420 and polydextrose, 10¹⁰ CFU/day+12 g/day.

The products for the study were provided in a sachet that the participant mixed with a 250 ml fruit smoothie once per day at the time of their liking for six months.

All randomized participants were 18-65 years old with a Body Mass Index (BMI, calculated as body weight [kg] divided by height [m] squared) between 28.0-34.9 and a waist-hip ratio of ≥0.88 for males and 0.83 for females. The most important exclusion criteria included diagnosed metabolic diseases or the use of related medications; use of laxatives, fibre supplements or probiotics in the previous 6 weeks; history of bariatric surgery; use of anti-obesity drugs in the previous 3 months, recent (past 2 months) or on-going use of antimicrobials; on-going or recent participation in a weight-loss program; weight change of 3 kg during previous 3 months; and pregnancy.

Recruitment and Study Populations

Before unblinding the study, 209 participants were selected from the 225 participants and were placed into an Intention-to-Treat (ITT) population. The ITT population contained all 209 participants who were assessed for any parameter after the baseline visit.

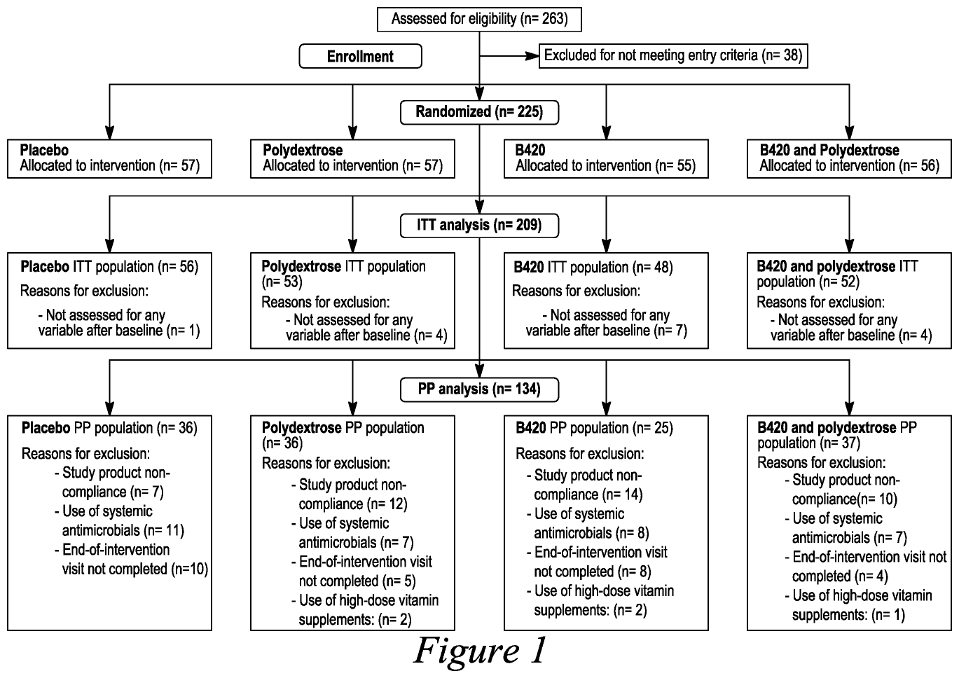

Of the 209 participants in the Intention-to-Treat (ITT) group, 134 participants completed the intervention period with at least 80% study product compliance, and did not use systemic antimicrobials or high-dose vitamin supplements during the intervention (Per protocol population (PP)). Therefore, the PP population better represents the efficacy of the product used in the study (FIG. 1).

FIG. 1 shows that before unblinding the study, participants were divided into an Intention-to-Treat (ITT) population and a Per Protocol (PP) population according to adherence to the study protocol. Several reasons may apply to a single excluded individual (n represents the number of people involved in each step of the process).

Dietary Intake Assessment

The participants filled in a 5-day food diary prior to the baseline, 2-month and end-of-intervention (6-month) clinic visits. Qualified nutritionists analyzed the food diary data with AivoDiet software (Aivo Finland Oy, Finland) using a national database of food ingredients and their compositions (Fineli, National Institute of Health and Welfare, Finland). Because not all study participants recorded the fruit smoothie vehicle in the food diary, data were recorded without the fruit smoothie. The energy content (130 kcal/day) of the fruit smoothie was later added to the energy intake data of all groups and all visits after baseline. The participant data for baseline body weight, height and age were used to calculate basal metabolic rate (BMR), as shown below:

BMR=655,0955+(9,5634*Body weight(kg))+(1,8496*Body height(cm))−(4,6756*Age in years) kcal/day Women:

BMR=66,4730+(13,7516*Body weight(kg))+(5,0033*Body height(cm))−(6.755*Age in years) kcal/day Men:

Basal metabolic rate is the amount of energy expended while at rest in a neutrally temperate environment, in the post-absorptive state. Food diaries with energy intake below 80% of the basal metabolic rate for women and 85% for men were regarded as underreported and consequently excluded from the analyses (FIG. 1).

Statistical Analysis

The mean change from baseline in all three active groups (groups taking B420, B420 and polydextrose together and polydextrose alone) was compared to placebo as an overall effect (one-way analysis of covariance, using baseline values as covariate). The three active groups were then compared to placebo separately using Dunnett's test, which corrects for multiple comparisons. All analyses were conducted with SAS analysis software, version 9.3. In the ITT population, missing observations were handled with the Last Observation Carried Forward method. In statistics, Dunnett's test is a multiple comparison procedure developed by Canadian statistician Charles Dunnett to compare each of a number of treatments with a single control. Multiple comparisons to a control are also referred to as many-to-one comparisons. The Last Observation Carried Forward method means that for missing values the latest measured value from a previous time point was used in the analysis. A P-value below 0.05 was considered statistically significant, meaning that the hypothesis of the compared observations being different is true with a 95% probability. A P-value above 0.05 does not prove that there was no difference, but rather there is not enough statistical power to draw conclusions with confidence.

Results

There was a statistically significant overall effect of the study products on energy intake in the PP population (P=0.0054, active groups vs. placebo) (Table 2), but not in the ITT population (P=0.23, active groups vs. placebo) (Table 1). However, differences in the ITT population showed a very similar pattern as in the PP population.

In the PP population, energy intake was statistically significantly reduced by B420 alone (−318.9 kcal/day, P=0.037, B420 vs. placebo) and the combination of B420 with polydextrose (−227 kcal/day, P=0.041), compared with placebo (−23.1 kcal/day) during the 6-month intervention. Polydextrose seemed to decrease energy intake (−200.6 kcal/day), but was not statistically significantly different from placebo (P=0.16, polydextrose vs. placebo) (Table 2).

Absolute fat intake was also statistically significantly different in the active groups compared to the placebo group in the PP population (P=0.008, active groups vs. placebo) (Table 4), but not in the ITT population (P=0.21, active groups vs. placebo), although changes in the ITT population did reflect those seen in the PP population (Table 3). This difference in fat intake was statistically significant in the group taking B420 alone (−21.6 g/day, P=0.03) with a similar trend in the combination of B420 with polydextrose (−10.1 g/day, P=0.11) and polydextrose alone (−11.6 g/day, P=0.17) groups compared to placebo (−2.2 g/day) (Table 4).

There was a borderline non-significant trend towards a decreased dietary proportion of fat in the active groups in the PP population (P=0.058, active groups vs. placebo) (Table 6). A “trend” means a P-value between 0.05-0.10 or sometimes, sometimes up to 0.15. There was no statistically significant overall difference in the ITT population (P=0.47, active groups vs. placebo) (Table 5), results in the ITT population seemed to reflect the changes seen in the PP population.

The decreased proportion of fat in the diet was mostly evident in the PP population in the group taking B420 only (−4.0%, P=0.11) compared to placebo (−0.6%) (Table 6). The other groups showed a smaller and statistically non-significant difference compared to placebo (polydextrose: −2.0%, P=0.39; B420 and polydextrose: −1.7%, P=0.52) Decreasing dietary fat intake is linked to a healthier lifestyle and may help reduce the risk of metabolic disorders.

The fact that differences were much greater in the PP population than in the ITT population indicates that the positive effect was due to the product used in the study. This is because the PP population included only those who were compliant with the protocol and used at least 80% of the study product, whereas the ITT population includes also those who were poorly complying and did not use the study product at all or in adequate amounts.

TABLE 1
Energy intake (kcal) in the Intention-to-Treat population

| | | Energy (kcal) | | | | | | | |
|---|---|---|---|---|---|---|---|---|---|
| Product | Visit | n | Mean | SD | Min | Q1 | Median | Q3 | Max |
| B420 | Baseline | 45 | 2169.4 | 442.2 | 1415.6 | 1958.9 | 2084.0 | 2389.0 | 2980.5 |
| | month 2 | 41 | 2253.2 | 479.3 | 1270.1 | 2042.2 | 2179.1 | 2478.7 | 3750.8 |
| | month 6 | 36 | 2039.5 | 550.9 | 1297.0 | 1601.2 | 2005.3 | 2324.7 | 3737.8 |
| B420 and | Baseline | 50 | 2077.8 | 625.8 | 1142.1 | 1692.0 | 1982.4 | 2141.4 | 4033.5 |
| polydextrose | month 2 | 47 | 1980.0 | 438.2 | 1261.6 | 1633.8 | 1959.6 | 2322.7 | 2771.0 |
| | month 6 | 41 | 1898.9 | 474.0 | 1159.2 | 1613.4 | 1873.4 | 2083.5 | 3599.1 |
| Polydextrose | Baseline | 50 | 2211.4 | 593.4 | 1280.9 | 1753.2 | 2149.7 | 2515.3 | 4545.2 |
| | month 2 | 50 | 2197.3 | 598.4 | 1345.9 | 1799.1 | 2097.9 | 2470.2 | 4606.1 |
| | month 6 | 49 | 2050.3 | 518.8 | 1259.7 | 1730.3 | 2027.7 | 2299.1 | 3537.6 |
| Placebo | Baseline | 53 | 2144.9 | 571.9 | 1293.7 | 1767.0 | 2010.6 | 2418.0 | 4005.5 |
| | month 2 | 51 | 2241.8 | 526.6 | 1356.7 | 1954.5 | 2115.6 | 2461.4 | 3948.6 |
| | month 6 | 49 | 2115.6 | 448.4 | 1384.3 | 1822.9 | 2065.4 | 2353.9 | 3542.8 |

| | | Change from baseline (kcal) | | | | | | | |
|---|---|---|---|---|---|---|---|---|---|
| Product | Visit | n | Mean | SD | Min | Q1 | Median | Q3 | Max |
| B420 | Baseline | 0 | . | . | . | . | . | . | . |
| | month 2 | 40 | 62.0 | 467.2 | −615.0 | −254.2 | −29.3 | 290.6 | 1681.2 |
| | month 6 | 35 | −190.0 | 473.3 | −952.0 | −543.9 | −255.3 | 34.1 | 1051.1 |
| B420 and | Baseline | 0 | . | . | . | . | . | . | . |
| polydextrose | month 2 | 46 | −113.7 | 523.7 | −1736 | −457.3 | −4.4 | 208.1 | 810.5 |
| | month 6 | 39 | −123.9 | 634.6 | −2160 | −289.0 | −51.0 | 220.6 | 1122.2 |
| Polydextrose | Baseline | 0 | . | . | . | . | . | . | . |
| | month 2 | 48 | 2.9 | 461.3 | −704.9 | −438.8 | 28.3 | 292.1 | 1435.8 |
| | month 6 | 48 | −160.5 | 466.4 | −1142 | −515.5 | −191.7 | 138.6 | 1093.5 |
| Placebo | Baseline | 0 | . | . | . | . | . | . | . |
| | month 2 | 48 | 87.8 | 576.2 | −1988 | −171.6 | 129.1 | 449.1 | 1162.0 |
| | month 6 | 46 | −4.4 | 539.2 | −1961 | −189.5 | 13.2 | 180.9 | 1813.0 |

No statistically significant differences. Only changes from baseline to month 6 were statistically compared.
n: number of observations; Mean: the average energy intake or change in energy intake calculated based on the number of participants in the corresponding group and corresponding visit displayed in columns “Product” and “Visit”. SD: Standard deviation; Min: Minimum value; Q1: First quartile; Q3: Third quartile; Max: Maximum value.

TABLE 2
Energy intake (kcal) in the Per Protocol population

| | | | | | | | | | | Change from |
|---|---|---|---|---|---|---|---|---|---|---|
| | | Energy (kcal) | | | | | | | | baseline (kcal) |
| Product | Visit | n | Mean | SD | Min | Q1 | Median | Q3 | Max | n |
| B420 | Baseline | 24 | 2203.7 | 380.6 | 1451.6 | 1997.1 | 2168.7 | 2453.9 | 2980.5 | 0 |
| | month 2 | 22 | 2133.7 | 383.7 | 1270.1 | 1812.0 | 2121.5 | 2478.7 | 2699.5 | 22 |
| | month 6 | 22 | 1904.7 | 371.4 | 1297.0 | 1587.7 | 1930.0 | 2210.1 | 2528.7 | 22 |
| B420 and | Baseline | 35 | 2092.3 | 643.2 | 1142.1 | 1661.9 | 1995.8 | 2141.4 | 4033.5 | 0 |
| polydextrose | month 2 | 34 | 1986.7 | 435.5 | 1261.6 | 1643.6 | 1932.6 | 2297.0 | 2771.0 | 33 |
| | month 6 | 31 | 1866.3 | 489.7 | 1243.6 | 1489.0 | 1870.8 | 2000.4 | 3599.1 | 29 |
| Polydextrose | Baseline | 33 | 2214.8 | 653.5 | 1280.9 | 1726.6 | 2234.3 | 2526.1 | 4545.2 | 0 |
| | month 2 | 35 | 2165.9 | 637.4 | 1367.2 | 1747.0 | 2092.8 | 2289.4 | 4606.1 | 33 |
| | month 6 | 34 | 2004.0 | 521.2 | 1259.7 | 1667.2 | 2018.1 | 2260.6 | 3537.6 | 33 |
| Placebo | Baseline | 33 | 2237.7 | 512.1 | 1397.5 | 1868.5 | 2159.1 | 2492.7 | 3838.8 | 0 |
| | month 2 | 34 | 2274.8 | 569.2 | 1455.8 | 1946.9 | 2246.3 | 2613.6 | 3948.6 | 31 |
| | month 6 | 36 | 2182.1 | 463.6 | 1384.3 | 1881.4 | 2096.5 | 2540.5 | 3542.8 | 33 |

| | | Change from baseline (kcal) | | | | | | |
|---|---|---|---|---|---|---|---|---|
| Product | Visit | Mean | SD | Min | Q1 | Median | Q3 | Max |
| B420 | Baseline | . | . | . | . | . | . | . |
| | month 2 | −115.3 | 253.6 | −443.6 | −326.5 | −203.7 | 42.4 | 486.5 |
| | month 6 | −318.9* | 303.1 | −787.0 | −543.9 | −319.5 | −147.2 | 519.0 |
| B420 and | Baseline | . | . | . | . | . | . | . |
| polydextrose | month 2 | −110.1 | 534.8 | −1736 | −395.3 | −17.3 | 208.1 | 628.4 |
| | month 6 | −227.0* | 640.5 | −2160 | −324.1 | −98.8 | 62.4 | 1122.2 |
| Polydextrose | Baseline | . | . | . | . | . | . | . |
| | month 2 | −15.1 | 425.3 | −704.9 | −455.4 | 26.1 | 249.2 | 1059.7 |
| | month 6 | −200.6 | 508.8 | −1142 | −682.3 | −191.0 | 105.9 | 1093.5 |
| Placebo | Baseline | . | . | . | . | . | . | . |
| | month 2 | 56.8 | 508.0 | −1534 | −182.1 | 60.1 | 364.4 | 1162.0 |
| | month 6 | −23.1 | 599.9 | −1961 | −189.5 | 2.5 | 148.3 | 1813.0 |

*= significant difference from Placebo (Dunnett's test, corrected for multiple comparisons). Only changes from baseline to month 6 were statistically compared.
n: number of observations; Mean: the average energy intake or change in energy intake calculated based on the number of participants in the corresponding group and corresponding visit displayed in columns “Product” and “Visit”. SD: Standard deviation; Min: Minimum value; Q1: First quartile; Q3: Third quartile; Max: Maximum value.

TABLE 3
Fat intake (g) in the Intention-to-Treat population

| | | Fat (g) | | | | | | | | Change from baseline (g) | | | | | | | |
|---|---|---|---|---|---|---|---|---|---|---|---|---|---|---|---|---|---|
| Product | Visit | n | Mean | SD | Min | Q1 | Median | Q3 | Max | n | Mean | SD | Min | Q1 | Median | Q3 | Max |
| B420 | Baseline | 45 | 92.8 | 24.1 | 43.4 | 80.7 | 88.7 | 105.8 | 151.1 | 0 | . | . | . | . | . | . | . |
| | month 2 | 41 | 91.6 | 27.0 | 45.2 | 72.9 | 88.4 | 108.3 | 169.9 | 40 | −2.6 | 24.9 | −48.2 | −18.0 | −5.6 | 15.8 | 59.6 |
| | month 6 | 36 | 79.1 | 25.5 | 43.3 | 57.6 | 69.0 | 98.6 | 126.2 | 35 | −16.8 | 23.5 | −62.2 | −32.1 | −18.0 | −7.1 | 38.9 |
| B420 and | Baseline | 50 | 86.4 | 31.7 | 33.8 | 66.6 | 78.5 | 98.2 | 178.7 | 0 | . | . | . | . | . | . | . |
| polydextrose | month 2 | 47 | 79.2 | 24.9 | 42.5 | 59.6 | 78.9 | 104.3 | 127.7 | 46 | −7.9 | 28.5 | −90.1 | −20.6 | 1.1 | 11.7 | 41.2 |
| | month 6 | 41 | 76.9 | 30.1 | 32.9 | 55.8 | 74.4 | 90.6 | 158.6 | 39 | −4.0 | 29.7 | −80.4 | −17.9 | −5.7 | 6.1 | 58.0 |
| Polydextrose | Baseline | 50 | 91.8 | 27.8 | 46.1 | 73.4 | 90.7 | 104.0 | 195.8 | 0 | . | . | . | . | . | . | . |
| | month 2 | 50 | 92.4 | 29.4 | 47.9 | 72.6 | 90.5 | 99.4 | 215.3 | 48 | 1.4 | 27.6 | −43.6 | −18.0 | −3.0 | 23.7 | 94.1 |
| | month 6 | 49 | 81.5 | 25.7 | 42.3 | 62.2 | 79.5 | 91.0 | 168.3 | 48 | −10.0 | 25.1 | −53.1 | −28.4 | −7.1 | 4.9 | 62.8 |
| Placebo | Baseline | 53 | 90.0 | 32.7 | 40.4 | 68.6 | 86.9 | 103.4 | 194.6 | 0 | . | . | . | . | . | . | . |
| | month 2 | 51 | 87.8 | 28.6 | 43.7 | 69.0 | 82.2 | 94.7 | 176.4 | 48 | −2.6 | 32.9 | −110.1 | −17.4 | 1.7 | 16.4 | 58.4 |
| | month 6 | 49 | 86.5 | 29.3 | 41.4 | 70.3 | 81.2 | 98.5 | 210.2 | 46 | −1.9 | 37.2 | −114.8 | −19.8 | −5.2 | 13.8 | 152.6 |

No statistically significant differences. Only changes from baseline to month 6 were statistically compared.
n: number of observations; Mean: the average energy intake or change in energy intake calculated based on the number of participants in the corresponding group and corresponding visit displayed in columns “Product” and “Visit”. SD: Standard deviation; Min: Minimum value; Q1: First quartile; Q3: Third quartile; Max: Maximum value.

TABLE 4
Fat intake (g) in the Per Protocol population

| | | Fat (g) | | | | | | | | Change from baseline (g) | | | | | | | |
|---|---|---|---|---|---|---|---|---|---|---|---|---|---|---|---|---|---|
| Product | Visit | n | Mean | SD | Min | Q1 | Median | Q3 | Max | n | Mean | SD | Min | Q1 | Median | Q3 | Max |
| B420 | Baseline | 24 | 93.5 | 21.6 | 45.1 | 81.9 | 91.0 | 105.7 | 146.1 | 0 | . | . | . | . | . | . | . |
| | month 2 | 22 | 86.9 | 26.3 | 45.2 | 64.8 | 84.0 | 105.8 | 136.6 | 22 | −9.7 | 18.4 | −45.1 | −20.2 | −12.1 | −0.9 | 37.5 |
| | month 6 | 22 | 73.6 | 21.7 | 43.3 | 57.0 | 67.5 | 88.8 | 116.5 | 22 | −21.6* | 18.8 | −62.2 | −33.7 | −21.1 | −9.5 | 14.9 |
| B420 and | Baseline | 35 | 87.3 | 31.9 | 33.8 | 66.3 | 81.0 | 110.4 | 162.7 | 0 | . | . | . | . | . | . | . |
| polydextrose | month 2 | 34 | 78.6 | 25.6 | 42.5 | 59.6 | 72.5 | 99.1 | 127.7 | 33 | −8.6 | 28.7 | −90.1 | −19.7 | 0.9 | 10.2 | 27.5 |
| | month 6 | 31 | 74.1 | 30.9 | 32.9 | 52.3 | 69.1 | 80.6 | 158.6 | 29 | −10.1 | 28.5 | −80.4 | −20.6 | −7.4 | 3.3 | 58.0 |
| Polydextrose | Baseline | 33 | 92.2 | 29.0 | 46.1 | 73.4 | 91.9 | 103.3 | 195.8 | 0 | . | . | . | . | . | . | . |
| | month 2 | 35 | 92.9 | 30.8 | 47.9 | 71.7 | 89.3 | 99.8 | 215.3 | 33 | 2.2 | 23.3 | −42.6 | −14.3 | −2.7 | 23.3 | 48.3 |
| | month 6 | 34 | 80.0 | 27.3 | 42.3 | 57.8 | 75.9 | 90.1 | 168.3 | 33 | −11.6 | 24.4 | −51.8 | −28.0 | −12.2 | 3.2 | 62.8 |
| Placebo | Baseline | 33 | 96.1 | 32.0 | 44.7 | 76.4 | 94.0 | 110.5 | 194.6 | 0 | . | . | . | . | . | . | . |
| | month 2 | 34 | 89.9 | 32.3 | 44.5 | 68.8 | 82.5 | 94.7 | 176.4 | 31 | −4.9 | 31.9 | −108.0 | −22.6 | −6.2 | 13.8 | 58.4 |
| | month 6 | 36 | 91.8 | 31.3 | 41.4 | 73.9 | 83.9 | 107.6 | 210.2 | 33 | −2.2 | 42.8 | −114.8 | −24.9 | −2.6 | 16.8 | 152.6 |

*= significant difference from Placebo (Dunnett's test, corrected for multiple comparisons) Only changes from baseline to month 6 were statistically compared.
n: number of observations; Mean: the average energy intake or change in energy intake calculated based on the number of participants in the corresponding group and corresponding visit displayed in columns “Product” and “Visit”. SD: Standard deviation; Min: Minimum value; Q1: First quartile; Q3: Third quartile; Max: Maximum value.

TABLE 5
Fat intake (% kcal) in the Intention-to-Treat population

| | | Fat intake (% of kcal) | | | | | | | |
|---|---|---|---|---|---|---|---|---|---|
| Product | Visit | n | Mean | SD | Min | Q1 | Median | Q3 | Max |
| B420 | Baseline | 45 | 38.3% | 5.0% | 26.0% | 35.0% | 38.4% | 41.5% | 47.2% |
| | month 2 | 41 | 36.3% | 5.9% | 22.9% | 32.3% | 36.1% | 40.8% | 50.0% |
| | month 6 | 36 | 34.8% | 6.1% | 24.5% | 30.3% | 34.1% | 37.9% | 48.7% |
| B420 and | Baseline | 50 | 37.1% | 6.2% | 25.5% | 32.5% | 36.9% | 41.1% | 55.3% |
| polydextrose | month 2 | 47 | 35.7% | 6.2% | 19.4% | 31.2% | 35.3% | 40.5% | 50.1% |
| | month 6 | 41 | 35.6% | 6.7% | 19.9% | 31.3% | 35.8% | 38.4% | 53.9% |
| Polydextrose | Baseline | 50 | 37.3% | 5.0% | 25.3% | 34.4% | 37.5% | 40.5% | 47.7% |
| | month 2 | 50 | 37.7% | 4.7% | 29.4% | 34.0% | 38.1% | 41.2% | 49.1% |
| | month 6 | 49 | 35.5% | 4.0% | 27.3% | 32.7% | 36.1% | 37.3% | 43.8% |
| Placebo | Baseline | 53 | 37.2% | 6.3% | 23.3% | 33.9% | 36.8% | 41.7% | 56.7% |
| | month 2 | 51 | 35.0% | 5.9% | 26.9% | 31.6% | 34.2% | 37.2% | 54.9% |
| | month 6 | 49 | 36.3% | 6.6% | 26.1% | 31.4% | 35.2% | 40.0% | 57.1% |

| | | Change from baseline (% of kcal) | | | | | | | |
|---|---|---|---|---|---|---|---|---|---|
| Product | Visit | n | Mean | SD | Min | Q1 | Median | Q3 | Max |
| B420 | Baseline | 0 | . | . | . | . | . | . | . |
| | month 2 | 40 | −2.2% | 6.2% | −14.3% | −6.3% | −2.2% | 1.4% | 11.0% |
| | month 6 | 35 | −3.8% | 6.2% | −15.5% | −7.9% | −4.1% | 0.2% | 14.1% |
| B420 and | Baseline | 0 | . | . | . | . | . | . | . |
| polydextrose | month 2 | 46 | −1.4% | 7.0% | −21.5% | −5.2% | −1.2% | 2.4% | 13.9% |
| | month 6 | 39 | −0.3% | 5.7% | −7.9% | −5.5% | −0.9% | 2.1% | 16.3% |
| Polydextrose | Baseline | 0 | . | . | . | . | . | . | . |
| | month 2 | 48 | 0.4% | 6.4% | −12.9% | −5.1% | −0.6% | 5.4% | 13.2% |
| | month 6 | 48 | −1.7% | 6.2% | −14.7% | −5.6% | −2.0% | 1.5% | 17.4% |
| Placebo | Baseline | 0 | . | . | . | . | . | . | . |
| | month 2 | 48 | −2.2% | 5.5% | −14.1% | −6.0% | −2.8% | 1.5% | 10.7% |
| | month 6 | 46 | −0.8% | 7.5% | −16.5% | −4.7% | −1.5% | 2.4% | 23.4% |

No statistically significant differences. Only changes from baseline to month 6 were statistically compared.
n: number of observations; Mean: the average energy intake or change in energy intake calculated based on the number of participants in the corresponding group and corresponding visit displayed in columns “Product” and “Visit”. SD: Standard deviation; Min: Minimum value; Q1: First quartile; Q3: Third quartile; Max: Maximum value.

TABLE 6
Fat intake (% kcal) in the Per Protocol population

| | | Fat intake (% of kcal) | | | | | | | |
|---|---|---|---|---|---|---|---|---|---|
| Product | Visit | n | Mean | SD | Min | Q1 | Median | Q3 | Max |
| B420 | Baseline | 24 | 38.0% | 4.8% | 27.9% | 34.8% | 37.6% | 41.4% | 47.2% |
| | month 2 | 22 | 36.3% | 6.6% | 22.9% | 31.7% | 36.9% | 41.5% | 50.0% |
| | month 6 | 22 | 34.5% | 6.0% | 24.5% | 30.1% | 33.9% | 38.4% | 46.2% |
| B420 and | Baseline | 35 | 37.2% | 6.4% | 25.6% | 32.5% | 37.3% | 40.7% | 55.3% |
| polydextrose | month 2 | 34 | 35.2% | 6.5% | 19.4% | 30.9% | 33.8% | 39.5% | 50.1% |
| | month 6 | 31 | 34.7% | 6.3% | 19.9% | 30.4% | 34.7% | 37.1% | 53.9% |
| Polydextrose | Baseline | 33 | 37.5% | 4.7% | 26.6% | 34.5% | 37.4% | 40.9% | 45.5% |
| | month 2 | 35 | 38.5% | 4.7% | 30.8% | 34.7% | 38.6% | 41.6% | 49.1% |
| | month 6 | 34 | 35.4% | 4.0% | 27.3% | 32.7% | 36.1% | 37.3% | 43.8% |
| Placebo | Baseline | 33 | 38.2% | 7.0% | 23.3% | 34.6% | 38.4% | 42.0% | 56.7% |
| | month 2 | 34 | 35.2% | 6.8% | 26.9% | 30.8% | 33.9% | 39.1% | 54.9% |
| | month 6 | 36 | 37.3% | 6.6% | 26.9% | 32.3% | 36.3% | 40.3% | 57.1% |
| | | Change from baseline (% of kcal) | | | | | | | |
| Product | Visit | n | Mean | SD | Min | Q1 | Median | Q3 | Max |
| B420 | Baseline | 0 | . | . | . | . | . | . | . |
| | month 2 | 22 | −2.4% | 5.6% | −14.0% | −6.3% | −2.1% | −0.1% | 10.2% |
| | month 6 | 22 | −4.0% | 5.9% | −15.5% | −8.1% | −3.2% | 1.4% | 3.9% |
| B420 and | Baseline | 0 | . | . | . | . | . | . | . |
| polydextrose | month 2 | 33 | −1.7% | 6.5% | −17.6% | −6.2% | −2.1% | 1.1% | 13.9% |
| | month 6 | 29 | −1.2% | 5.4% | −7.9% | −5.5% | −2.0% | 1.4% | 16.3% |
| Polydextrose | Baseline | 0 | . | . | . | . | . | . | . |
| | month 2 | 33 | 0.9% | 6.1% | −12.9% | −5.1% | 0.6% | 5.1% | 12.3% |
| | month 6 | 33 | −2.0% | 5.6% | −11.3% | −5.7% | −3.2% | 1.4% | 13.0% |
| Placebo | Baseline | 0 | . | . | . | . | . | . | . |
| | month 2 | 31 | −2.7% | 6.2% | −14.1% | −7.6% | −2.9% | 0.6% | 10.7% |
| | month 6 | 33 | −0.6% | 8.3% | −16.5% | −4.7% | −1.0% | 3.4% | 23.4% |

No statistically significant differences. Only changes from baseline to month 6 were statistically compared.
n: number of observations; Mean: the average energy intake or change in energy intake calculated based on the number of participants in the corresponding group and corresponding visit displayed in columns “Product” and “Visit”. SD: Standard deviation; Min: Minimum value; Q1: First quartile; Q3: Third quartile; Max: Maximum value.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1-11. (canceled)

12. A method for reducing food, energy and/or fat intake comprising administering to a mammal a bacterium of the genus Bifidobacterium or a mixture thereof, wherein:
the administration of the bacterium of the genus Bifidobacterium reduces the food, energy and/or fat intake in the mammal; and
the bacterium of the genus Bifidobacterium comprises Bifidobacterium animalis ssp. lactis strain 420 (B420).

13-14. (canceled)

15. The method according to claim 12, wherein the bacterium is used in combination with one or more fibres and/or prebiotics.

16. The method according to claim 15, wherein the one or more fibres and/or prebiotic comprise(s) polydextrose.

17. A method according to claim 12, wherein the bacterium is administered to the mammal in the form of a food product, a dietary supplement or a pharmaceutically acceptable formulation.

18. (canceled)

19. The method according to claim 17, wherein the bacterium is used in combination with one or more fibres and/or prebiotics.

20. The method according to claim 19, wherein the one or more fibres and/or prebiotic comprise(s) polydextrose.

21. The method according to claim 17, wherein the bacterium is administered to the mammal in the form of a medical food product.

22. The method according to claim 21, wherein the bacterium is used in combination with one or more fibres and/or prebiotics.

23. The method according to claim 22, wherein the one or more fibres and/or prebiotic comprise(s) polydextrose.

24. The method according to claim 17, wherein the bacterium is administered to the mammal in the form of a medicament.

25. The method according to claim 24, wherein the bacterium is used in combination with one or more fibres and/or prebiotics.

26. The method according to claim 25, wherein the one or more fibres and/or prebiotic comprise(s) polydextrose.
